(19) United States
(12) Patent Application Publication
(10) Pub. No.: US 2025/0213742 A1
(43) Pub. Date: Jul. 3, 2025

(54) APPARATUS FOR CONTROLLED EMISSION OF SCENTS

(71) Applicant: Brand3D, Inc., Tampa, FL (US)

(72) Inventor: Hans Hesselholt Henne HANSEN, Tampa, FL (US)

(21) Appl. No.: 19/004,691

(22) Filed: Dec. 30, 2024

Related U.S. Application Data
(60) Provisional application No. 63/615,869, filed on Dec. 29, 2023.

(51) Int. Cl. A61L 9/12

(52) U.S. Cl. CPC A61L 9/122; A61L 9/125; A61L 2209/11; A61L 2209/133

(57) ABSTRACT

An apparatus for controlled emission of scents includes a housing, a set of scent tubes within the housing, each scent tube configured to house a fragrance cartridge, a blower configured to blow air towards at least one scent tube of the set of scent tubes, a blower sliding mechanism configured to move the blower adjacent to a selected scent tube, and a blower holding mechanism configured to secure the blower in alignment with the selected scent tube. The apparatus enables precise and selective scent emission by allowing the blower to be positioned adjacent to a desired scent tube and securely aligned to disperse the selected fragrance.

### CROSS-REFERENCE TO RELATED APPLICATIONS

This application claims priority from U.S. provisional application No. 63/615,869 filed on Dec. 29, 2023.

### FIELD OF INVENTION

The present disclosure relates to scent emission devices, and more particularly to an apparatus for controlled emission of scents using a set of scent tubes, at least one blower, and associated mechanisms.

### BACKGROUND

The use of scents in various environments, such as retail spaces, hospitality settings, and residential areas, has become increasingly important for creating desired atmospheres and enhancing overall experiences. Scent dispersal systems aim to influence ambiance by diffusing fragrances into the surrounding air. However, existing systems often face challenges in achieving precise control over scent emission and targeted dispersion.

Many current scent emission devices require manual effort to release fragrances and are typically limited to dispersing a single scent. Some automated systems employ trigger mechanisms that disperse scents at regular time intervals, but these lack the ability to precisely regulate the timing, intensity, and distribution of fragrances in a given area.

The inefficiencies in existing scent dispersal systems can lead to irregular or uneven fragrance distribution, potentially diminishing the desired effect or failing to create a consistent olfactory experience throughout an environment. Additionally, the lack of precise control over scent emission may result in wastage of fragrance materials, impacting both cost-effectiveness and sustainability of scent-based strategies.

Furthermore, maintaining control over the intensity and timing of fragrances while ensuring uniform aroma distribution across various environments presents a significant challenge for current systems. This highlights the need for more sophisticated and accurate methods of fragrance dispersion that can offer greater flexibility and precision in scent emission control.

As the importance of creating immersive and tailored sensory experiences continues to grow across various industries, there is an increasing demand for advanced scent emission technologies that can overcome the limitations of existing systems and provide more refined control over fragrance dispersion.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, an apparatus for controlled emission of scents is provided. The apparatus includes a set of scent tubes, a blower, a blower sliding mechanism, a blower holding mechanism, and a housing. The set of scent tubes is configured to provide a housing for fragrance emitting rods or fragrance cartridges. The blower is configured to blow air towards at least one scent tube of the set of scent tubes, causing fragrance from the fragrance emitting rods inside the scent tube to be dispersed in the environment. The blower sliding mechanism and the blower holding mechanism are configured to place the blower adjacent to the scent tube having a selected fragrance. The housing is configured to enclose the entire structure of the apparatus.

According to other aspects of the present disclosure, the apparatus may include one or more of the following features. The apparatus may include a fragrance selection mechanism, which may be at least one of a set of physical buttons, a graphical user interface, and a microprocessor configured to receive data from at least one of a web interface and a mobile application. The set of scent tubes may be cylindrical chambers that are carved in the housing and equidistant to each other. The set of scent tubes may have an adjustable radius to accommodate fragrance cartridges of different sizes. Each scent tube of the set of scent tubes may have a locking mechanism to lock a fragrance cartridge in the scent tube. The locking mechanism may be at least one of a spring loaded clamp mechanism, a magnetic holder mechanism, a twist and lock mechanism, an adjustable clamp mechanism, a friction fit mechanism, and a bayonet mounting mechanism.

The apparatus may include an automated system for inserting the fragrance cartridges in the set of scent tubes. The automated system may include one or more sensors to measure the quantity of fragrance available in the fragrance cartridge and may be configured to replace a fragrance cartridge when the quantity of fragrance is under a predefined threshold. The apparatus may include an alert system to generate an alert for a human to replace the fragrance cartridge when the quantity of fragrance in the fragrance cartridge falls under a predefined threshold.

The blower may include a concentration adjustment mechanism to control the amount of fragrance to be emitted. The apparatus may include a leakage prevention mechanism installed in the set of scent tubes to prevent leakage of fragrance from a fragrance cartridge when the blower is not aligned with the fragrance cartridge. The blower sliding mechanism may include an automatic sliding unit comprising an electronically controlled motor. The blower holding mechanism may include at least one of one or more notches and one or more sloping structures that may direct the blower to align with the scent tube.

The apparatus may be programmable and may include a processor connected to a memory. The memory may have instructions stored for autonomous operation of the apparatus, including recording usage data, training a machine learning algorithm based on the usage data, and receiving feedback from a user to improve the machine learning algorithm.

The foregoing general description of the illustrative embodiments and the following detailed description thereof are merely exemplary aspects of the teachings of this disclosure and are not restrictive.

### BRIEF DESCRIPTION OF FIGURES

FIG. 1 illustrates a schematic representation of an apparatus for controlled emission of scents, according to aspects of the present disclosure.

FIG. 2 depicts a three-dimensional model of the apparatus for controlled emission of scents, according to an embodiment.

FIG. 3 shows another three-dimensional model of the apparatus for controlled emission of scents, according to aspects of the present disclosure.

FIG. 4 illustrates an alternate embodiment of the apparatus for controlled emission of scents, in accordance with example embodiments.

FIG. 5 depicts a flowchart illustrating a method 500 for controlled emission of scents, in accordance with example embodiments.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure provides an apparatus designed for the controlled emission of scents. This apparatus may include a set of scent tubes, a blower, a blower sliding mechanism, a blower holding mechanism, and a housing. The scent tubes may house fragrance emitting rods or fragrance cartridges, and the blower may direct air towards at least one scent tube, causing the fragrance from the rods or cartridges within the tube to disperse into the environment. The blower sliding mechanism and the blower holding mechanism may work together to position the blower adjacent to the scent tube containing the selected fragrance. The housing may enclose the entire structure of the apparatus. In some cases, the apparatus may also include a fragrance selection mechanism, a concentration adjustment mechanism, a leakage prevention mechanism, and an automated system for inserting fragrance cartridges into the scent tubes. This apparatus may offer enhanced control over scent emission, potentially improving the precision and efficiency of fragrance dispersion in various environments.

In some aspects, the apparatus for controlled emission of scents may incorporate a network architecture to enhance its functionality, connectivity, and control capabilities. This network architecture may enable remote management, data collection, and integration with other smart systems.

The network architecture may include a central control unit. The apparatus may contain a central processing unit that acts as the main controller. This unit may be equipped with a microprocessor, memory, and network interfaces. It may run the core software, including the machine learning algorithms, and coordinate all functions of the scent emission system.

In some implementations, the apparatus may utilize a Local Area Network (LAN). Within the apparatus, components such as the blowers, sensors, and fragrance cartridge mechanisms may be connected via a local area network. This may be implemented using technologies such as Ethernet, Wi-Fi, or specialized protocols like I2C or SPI for internal communication.

The apparatus may include wireless connectivity features. In some cases, the apparatus may incorporate Wi-Fi and/or Bluetooth modules to enable wireless communication. Wi-Fi may allow the device to connect to the internet and local networks, while Bluetooth may facilitate direct connections with nearby devices such as smartphones or tablets.

Internet connectivity may be a key feature of the network architecture. Through its Wi-Fi capability, the apparatus may connect to the internet. This may enable cloud-based services, remote management, and software updates.

In some aspects, the system may utilize cloud integration. The apparatus may leverage cloud services for data storage, advanced analytics, and remote access. This may allow for offloading computationally intensive tasks and providing scalable storage for usage data and user profiles.

The network architecture may include a mobile application interface. A dedicated mobile application may be developed to interface with the apparatus. This app may communicate with the device either directly through Bluetooth or via the internet, allowing users to control the system, receive notifications, and view analytics.

In some implementations, the apparatus may include a web server. The apparatus may incorporate an embedded web server, allowing users to access a web-based interface for control and monitoring through any web browser. This may provide an alternative to the mobile app for device management.

The network architecture may feature an API layer. An Application Programming Interface (API) may be implemented to allow integration with third-party systems and services. This may enable the apparatus to be part of a larger smart home or building management ecosystem.

Security protocols may be an integral part of the network architecture. The network architecture may incorporate robust security measures, including encryption for data transmission, secure boot processes, and authentication mechanisms to protect user data and prevent unauthorized access.

In some aspects, the apparatus may support mesh networking capability. In scenarios where multiple scent emission devices are deployed in a large space, the apparatus may support mesh networking. This may allow devices to communicate with each other and extend the network range without requiring individual internet connections for each device.

The system may incorporate IoT protocol support. The apparatus may support common Internet of Things (IoT) protocols such as MQTT or CoAP, facilitating efficient communication between devices and servers, especially in low-bandwidth or unreliable network conditions.

This network architecture may provide a flexible and scalable framework for the scent emission system, enabling advanced features such as remote management, data-driven decision making, and integration with smart building systems.

In some aspects, the apparatus for controlled emission of scents may be designed to enhance the ambiance of various settings such as retail, hospitality, and residential spaces by dispersing selected fragrances in a controlled manner. The apparatus may include several key components that work together to achieve this purpose.

One of these components may be a set of scent tubes. These scent tubes may serve as a housing for fragrance emitting rods or fragrance cartridges. In some cases, the scent tubes may be cylindrical chambers, while in other cases, they may have different shapes such as cubical or rectangular. The scent tubes may also be adjustable to accommodate fragrance cartridges of different sizes or shapes.

Another key component of the apparatus may be a blower. The blower may be designed to blow air towards at least one scent tube from the set of scent tubes. This air flow may cause the fragrance from the fragrance emitting rods or cartridges inside the scent tube to be dispersed into the environment. In some cases, the blower may include a concentration adjustment mechanism to control the amount of fragrance to be emitted.

The apparatus may also include a blower sliding mechanism and a blower holding mechanism. The blower sliding mechanism may allow the blower to be placed adjacent to the scent tube containing the selected fragrance. The blower holding mechanism may ensure the alignment of the blower with the scent tubes and secure the positioning of the blower during operation.

The entire structure of the apparatus may be enclosed in a housing. The housing may serve multiple purposes such as preventing the loss of fragrance from the fragrance emitting rods, preventing dust from settling in the mechanisms of the apparatus, and preventing damage to any part of the apparatus.

In some cases, the apparatus may also include additional features such as a fragrance selection mechanism, a leakage prevention mechanism, and an automated system for inserting fragrance cartridges into the scent tubes. These features may further enhance the functionality and efficiency of the apparatus.

In some aspects, the set of scent tubes 102 may be designed with an adjustable radius. This feature may allow the scent tubes 102 to accommodate fragrance cartridges of varying sizes. For instance, the radius of the scent tubes 102 may be increased or decreased to fit larger or smaller fragrance cartridges, respectively. This adjustability may provide flexibility in the types and sizes of fragrance cartridges that can be used with the apparatus, potentially expanding the range of fragrances that can be emitted.

In some cases, the scent tubes 102 may have different shapes. While cylindrical chambers may be used in some embodiments, other shapes such as cubical, rectangular, or other polygonal shapes may also be used in other embodiments. The shape of the scent tubes 102 may be selected based on various factors, such as the shape of the fragrance cartridges to be housed, the available space within the housing 110, or the desired aesthetic appearance of the apparatus. Regardless of the shape, the scent tubes 102 may be designed to securely house the fragrance emitting rods or cartridges and facilitate the controlled emission of scents.

In some embodiments, the scent tubes 102 may be designed to securely hold the fragrance emitting rods or cartridges in place during operation of the apparatus. This may be achieved through various mechanisms, such as a locking mechanism or a friction fit mechanism. These mechanisms may ensure that the fragrance emitting rods or cartridges are tightly held within the scent tubes 102, preventing them from moving or falling out during operation of the apparatus. This may contribute to the efficient and controlled emission of scents from the apparatus.

In some aspects, the blower 104 may be a key component of the apparatus for controlled emission of scents. The blower 104 may be designed to blow air towards at least one scent tube from the set of scent tubes 102. This air flow may cause the fragrance from the fragrance emitting rods or cartridges inside the scent tube to be dispersed into the environment. The blower 104 may be strategically positioned adjacent to the scent tube containing the selected fragrance, ensuring that the desired scent is emitted into the environment.

In some cases, the blower 104 may include a concentration adjustment mechanism. This mechanism may be designed to control the amount of fragrance to be emitted based on various inputs. For instance, the concentration adjustment mechanism may receive an input regarding the type of fragrance, the concentration of the fragrance, the size of the environment, or the location of the apparatus 100 in the environment. Based on these inputs, the concentration adjustment mechanism may calculate a fan rotation speed for the fan in the blower 104 and a fan rotation time. The fan rotation speed may correspond to the amount of fragrance to be dispersed from the cartridge per second, while the fan rotation time may correspond to the duration for which the blower 104 should be on. This feature may allow for precise control over the intensity and timing of the fragrance emission, potentially enhancing the overall scent experience in the environment.

In some embodiments, the blower 104 may be designed to work with multiple scent tubes 102 simultaneously. This may allow for the mixing of different fragrances and the creation of unique scent combinations. In such cases, the concentration adjustment mechanism may be designed to control the amount of each fragrance to be emitted, allowing for the creation of custom scent profiles.

In other cases, the blower 104 may be designed to work with a single scent tube 102 at a time. This may allow for the emission of a single, distinct fragrance into the environment. The concentration adjustment mechanism in these cases may be designed to control the intensity of the fragrance emission, allowing for the creation of a consistent and uniform scent experience in the environment.

In some aspects, the apparatus may include a blower sliding mechanism 106. This mechanism may be designed to allow the blower 104 to slide along a predefined track, enabling it to be positioned adjacent to the scent tube containing the selected fragrance. The blower sliding mechanism 106 may include an adjustment knob that protrudes from the housing 110. This knob may be manually pushed or pulled to move the blower 104 along the track. This manual operation may provide a user with direct control over the positioning of the blower 104, allowing them to select the desired fragrance for emission.

In some cases, the blower sliding mechanism 106 may include an automatic sliding unit. This unit may comprise an electronically controlled motor that moves the blower 104 along the predefined track. The electronically controlled motor may receive input from a fragrance selection mechanism, which may be a set of physical buttons, a graphical user interface, a web interface, or a mobile application. Based on this input, which may include a fragrance selection or a combination of fragrances, the electronically controlled motor may automatically position the blower 104 adjacent to the corresponding scent tube. This automatic operation may provide a user with a convenient and efficient way to control the emission of scents from the apparatus.

In other embodiments, the blower sliding mechanism 106 may include both manual and automatic options for positioning the blower 104. This dual-mode operation may provide a user with flexibility in controlling the emission of scents from the apparatus. For instance, the user may choose to manually position the blower 104 for a specific scent emission, or they may choose to use the automatic mode for regular or scheduled scent emissions. This flexibility may enhance the usability and functionality of the apparatus.

In some aspects, the apparatus may include a blower holding mechanism 108. This mechanism may be designed to ensure the proper alignment of the blower 104 with the scent tubes 102 and secure the positioning of the blower 104 during operation. The blower holding mechanism 108 may include one or more notches or sloping structures that guide the blower 104 to align with a scent tube. This mechanism may create a resistance for the sliding mechanism, causing it to stop at positions where the blower 104 is correctly aligned with the scent tubes 102. This feature may contribute to the precise and efficient emission of scents from the apparatus.

In some cases, the blower holding mechanism 108 may include a magnetic system. This system may include one or more magnets or electromagnets that pull the blower 104 towards a position on the predefined track. This magnetic attraction may help to align the blower 104 with a scent tube from the set of scent tubes 102. The use of a magnetic system may provide a smooth and precise movement of the blower 104 along the track, enhancing the accuracy of the scent emission.

In other embodiments, the blower holding mechanism 108 may include other types of systems or mechanisms for aligning and securing the blower 104. These may include, for example, mechanical latches, spring-loaded clips, or friction-based systems. Regardless of the specific type of system or mechanism used, the blower holding mechanism 108 may be designed to ensure the proper alignment and secure positioning of the blower 104, contributing to the controlled and efficient emission of scents from the apparatus.

The housing 110 of the apparatus may serve as an enclosure for the various components of the apparatus, including the set of scent tubes 102, the blower 104, the blower sliding mechanism 106, and the blower holding mechanism 108. In some aspects, the housing 110 may be designed to prevent the loss of fragrance from the fragrance emitting rods or cartridges housed within the scent tubes 102. This may be particularly beneficial in scenarios where the apparatus is not in operation, as it may help to conserve the fragrance materials and extend the lifespan of the fragrance cartridges.

In some cases, the housing 110 may also serve to protect the mechanisms of the apparatus from dust and other environmental contaminants. This may help to maintain the cleanliness and functionality of the apparatus, potentially reducing the need for frequent maintenance or cleaning. The housing 110 may be designed to be easily removable or openable for access to the internal components of the apparatus, facilitating maintenance and replacement of components as needed.

In other aspects, the housing 110 may also serve to protect the components of the apparatus from damage. For instance, the housing 110 may be made from a durable material such as metal, plastic, or a composite material, which may provide a degree of physical protection for the internal components. The housing 110 may also be designed to withstand various environmental conditions, such as changes in temperature or humidity, further enhancing the durability and longevity of the apparatus.

In some embodiments, the housing 110 may be designed with aesthetic considerations in mind. For instance, the housing 110 may be available in various colors, finishes, or designs to match the decor of the environment in which the apparatus is to be used. The housing 110 may also be designed with a compact and sleek form factor, allowing it to be discreetly placed in various locations within an environment.

In other cases, the housing 110 may include features such as vents or openings to facilitate the dispersion of the emitted scents into the environment. These vents or openings may be strategically positioned to direct the flow of the emitted scents, potentially enhancing the distribution and reach of the scents within the environment. The design and placement of these vents or openings may be adjustable based on the specific requirements of the environment or the preferences of the user.

In some aspects, the apparatus may include a fragrance selection mechanism. This mechanism may provide a user with the ability to select a specific fragrance or a combination of fragrances for emission from the apparatus. The fragrance selection mechanism may include various types of user interfaces or control options, depending on the specific design of the apparatus.

In some cases, the fragrance selection mechanism may include a set of physical buttons. These buttons may be located on the housing of the apparatus and may be labeled or color-coded to correspond to different fragrances or combinations of fragrances. A user may select a fragrance by pressing the corresponding button, causing the blower to align with the scent tube containing the selected fragrance.

In other cases, the fragrance selection mechanism may include a graphical user interface (GUI). The GUI may be displayed on a screen located on the housing of the apparatus or on a separate device such as a computer or a mobile device. The GUI may display a list or a grid of available fragrances, and a user may select a fragrance by clicking or tapping on the corresponding icon or button on the GUI. The GUI may also provide additional options or controls for adjusting the intensity or timing of the fragrance emission.

In yet other cases, the fragrance selection mechanism may include remote control options. For instance, the apparatus may be configured to receive data from a web interface or a mobile application. A user may use the web interface or the mobile application to select a fragrance, adjust the intensity or timing of the fragrance emission, or schedule automatic fragrance emissions at specific times. The web interface or the mobile application may communicate with the apparatus via a wired or wireless connection, allowing the user to control the apparatus from a distance.

In some embodiments, the fragrance selection mechanism may include a combination of the above options. For instance, the apparatus may include physical buttons for quick and easy fragrance selection, a GUI for more detailed control over the fragrance emission, and a web interface or a mobile application for remote control. This multi-modal control may provide a user with flexibility and convenience in controlling the emission of scents from the apparatus.

In some aspects, each scent tube of the set of scent tubes 102 may be equipped with a locking mechanism. This locking mechanism may be designed to secure a fragrance cartridge within the scent tube, ensuring that the cartridge is tightly held during the operation of the apparatus. The locking mechanism may take various forms, depending on the specific design of the apparatus and the fragrance cartridges.

In some cases, the locking mechanism may be a spring-loaded clamp mechanism. This type of mechanism may use the force of a spring to clamp the fragrance cartridge in place within the scent tube. The spring-loaded clamp mechanism may provide a secure hold on the cartridge while allowing for easy insertion and removal of the cartridge.

In other cases, the locking mechanism may be a magnetic holder mechanism. This type of mechanism may use one or more magnets to attract and hold the fragrance cartridge in place within the scent tube. The magnetic holder mechanism may provide a secure hold on the cartridge while allowing for easy insertion and removal of the cartridge.

In yet other cases, the locking mechanism may be a twist and lock mechanism. This type of mechanism may involve a threaded portion on the fragrance cartridge and a corresponding threaded portion within the scent tube. The fragrance cartridge may be inserted into the scent tube and then twisted to engage the threads and lock the cartridge in place.

In some embodiments, the locking mechanism may be an adjustable clamp mechanism. This type of mechanism may include a clamp that can be adjusted to fit fragrance cartridges of different sizes. The adjustable clamp mechanism may provide a secure hold on the cartridge while allowing for the use of cartridges of varying sizes.

In other embodiments, the locking mechanism may be a friction fit mechanism. This type of mechanism may involve a snug fit between the fragrance cartridge and the interior of the scent tube. The friction between the cartridge and the tube may hold the cartridge in place.

In yet other embodiments, the locking mechanism may be a bayonet mounting mechanism. This type of mechanism may involve a slot in the scent tube and a corresponding projection on the fragrance cartridge. The cartridge may be inserted into the tube and then twisted to engage the projection with the slot, locking the cartridge in place.

These various locking mechanisms may provide secure and reliable ways to hold the fragrance cartridges within the scent tubes, contributing to the efficient and controlled emission of scents from the apparatus. In some cases, the specific type of locking mechanism used may depend on factors such as the design of the fragrance cartridges, the materials used in the cartridges and the scent tubes, and the specific requirements of the user or the environment.

In some aspects, the apparatus may include an automated system for inserting the fragrance cartridges into the set of scent tubes 102. This automated system may be designed to reduce the need for manual intervention in the process of inserting and replacing the fragrance cartridges, potentially enhancing the efficiency and convenience of the apparatus.

The automated system may include one or more sensors designed to measure the quantity of fragrance available in the fragrance cartridge. These sensors may be located within the scent tubes 102 or in close proximity to the fragrance cartridges. In some cases, the sensors may use optical, chemical, or other types of sensing technologies to detect the quantity of fragrance in the cartridges. The sensors may send signals to a control unit in the apparatus, which may interpret the signals and determine the quantity of fragrance remaining in the cartridges.

In some embodiments, the automated system may also include a set of robotic arms designed to replace the fragrance cartridges in the scent tubes 102. The robotic arms may be controlled by the control unit and may be designed to grasp and manipulate the fragrance cartridges. The robotic arms may be capable of performing various tasks, such as removing a used fragrance cartridge from a scent tube, selecting a new fragrance cartridge from a storage area, and inserting the new cartridge into the scent tube.

In some cases, the automated system may be configured to replace a fragrance cartridge when the quantity of fragrance in the cartridge falls below a predefined threshold. This threshold may be set based on various factors, such as the desired intensity of the fragrance emission, the size of the environment, or the preferences of the user. When the quantity of fragrance in a cartridge falls below the threshold, the control unit may activate the robotic arms to replace the cartridge with a new one.

In other embodiments, the automated system may be designed to insert and replace fragrance cartridges based on a schedule or a set of rules defined by the user. For instance, the user may program the apparatus to replace the fragrance cartridges at specific times, or to alternate between different fragrances at different times of the day. The control unit may use this information to control the operation of the robotic arms and the insertion and replacement of the fragrance cartridges.

In yet other cases, the automated system may be designed to learn and adapt to the usage patterns of the user. For instance, the control unit may include a machine learning algorithm that analyzes the usage data collected by the sensors and adjusts the operation of the automated system accordingly. This feature may allow the apparatus to automatically adjust the timing and frequency of the fragrance cartridge replacement based on the user's habits and preferences, potentially enhancing the user experience and the efficiency of the apparatus.

In some aspects, the apparatus may include an alert system designed to notify a user when the fragrance cartridges need to be replaced. This alert system may be particularly useful in scenarios where the fragrance cartridges are manually inserted and replaced by a user. The alert system may generate alerts based on various factors, such as the quantity of fragrance remaining in the cartridges, the frequency of use of the apparatus, or the preferences of the user.

In some cases, the alert system may generate an auditory alert. This alert may be a sound or a series of sounds emitted by a speaker or a buzzer in the apparatus. The sound may be designed to be easily distinguishable from other sounds in the environment, ensuring that the user is aware of the need to replace the fragrance cartridges.

In other cases, the alert system may generate a visual alert. This alert may be a light or a series of lights emitted by a light source, such as a Light Emitting Diode (LED), on the housing of the apparatus. The light may be of a specific color or pattern to indicate the need for cartridge replacement. In some embodiments, the light may blink or flash to attract the attention of the user.

In yet other cases, the alert system may send a notification to a user device. This notification may be sent via a wired or wireless connection to a device such as a computer, a tablet, or a mobile phone. The notification may include information about the need to replace the fragrance cartridges, and may also include instructions or guidance on how to perform the replacement.

In some embodiments, the alert system may be designed to generate alerts at specific times or intervals. For instance, the alert system may generate an alert when the quantity of fragrance in a cartridge falls below a predefined threshold. This threshold may be set based on the desired intensity of the fragrance emission, the size of the environment, or the preferences of the user. The alert system may also generate alerts at regular intervals, such as once a day or once a week, to remind the user to check the fragrance cartridges and replace them if necessary.

In some aspects, the apparatus may include a leakage prevention mechanism. This mechanism may be designed to prevent the loss of fragrance from the fragrance cartridges when the blower is not aligned with the scent tube containing the cartridge. The leakage prevention mechanism may take various forms, depending on the specific design of the apparatus and the fragrance cartridges.

In some cases, the leakage prevention mechanism may be a rubber membrane. This membrane may be installed in the set of scent tubes and may be designed to open and close in response to the alignment of the blower with the scent tube. When the blower is aligned with a scent tube, the rubber membrane may open to allow the flow of air from the blower into the scent tube. When the blower is not aligned with the scent tube, the rubber membrane may close to prevent the escape of fragrance from the cartridge.

In other cases, the leakage prevention mechanism may be a one-way valve. This valve may be installed in the set of scent tubes and may be designed to allow the flow of air from the blower into the scent tube, but not in the opposite direction. This feature may prevent the escape of fragrance from the cartridge when the blower is not in operation.

In yet other cases, the leakage prevention mechanism may be a gravity-controlled flap. This flap may be installed in the set of scent tubes and may be designed to open when blown on by the blower and close when the blower is not in operation. The gravity-controlled flap may provide a simple and effective way to prevent the loss of fragrance from the cartridges.

In some aspects, the apparatus may include a set of cartridge caps attached to the scent tubes using a hinge mechanism. These cartridge caps may be designed to cover the ends of the fragrance cartridges away from the blower when the blower is not in operation. The hinge mechanism may allow the cartridge caps to be easily opened and closed, providing a convenient way to prevent the loss of fragrance from the cartridges. In some cases, the cartridge caps may be manually operated by a user. In other cases, the cartridge caps may be automatically operated by the apparatus, for instance, in response to the alignment of the blower with a scent tube.

In some aspects, the apparatus may include a programmable system for autonomous operation. This programmable system may include a processor and a memory. The memory may store instructions for controlling the operation of the apparatus, including the emission of scents, the movement of the blower, and the replacement of the fragrance cartridges. The processor may execute these instructions, controlling the various components of the apparatus to achieve the desired scent emission.

In some cases, the programmable system may include machine learning capabilities. The processor may be configured to execute a machine learning algorithm that learns from the usage data collected by the apparatus. This usage data may include information such as the frequency of each fragrance used, the timing of the scent emissions, and the preferences of the user. The machine learning algorithm may analyze this data and adjust the operation of the apparatus accordingly. For instance, the algorithm may learn to automatically select a fragrance and emit the scent at a particular time of the day based on the user's habits and preferences.

In other cases, the programmable system may include a feedback mechanism. The feedback mechanism may allow a user to provide feedback on the operation of the apparatus, such as the intensity of the fragrance emission, the timing of the scent emissions, or the selection of the fragrance. The processor may receive this feedback and adjust the operation of the apparatus accordingly. This feedback mechanism may enhance the adaptability of the apparatus, allowing it to better meet the needs and preferences of the user.

In yet other cases, the programmable system may include a scheduling mechanism. The scheduling mechanism may allow a user to program the apparatus to emit scents at specific times or intervals. The processor may receive this scheduling information and control the operation of the apparatus accordingly. This scheduling mechanism may provide a user with greater control over the scent emission, allowing them to create a customized scent experience in the environment.

In some embodiments, the programmable system may include a remote control mechanism. The remote control mechanism may allow a user to control the operation of the apparatus from a distance, for instance, via a web interface or a mobile application. The processor may receive control signals from the remote control mechanism and adjust the operation of the apparatus accordingly. This remote control mechanism may provide a user with convenience and flexibility in controlling the scent emission from the apparatus.

In some aspects, the apparatus may include multiple blowers, each aligned with a scent tube. This configuration may allow for simultaneous emission of multiple fragrances, potentially creating a unique scent combination in the environment. Each blower in the plurality of blowers may be independently controlled, allowing for precise control over the intensity and timing of each fragrance emission. In some cases, each blower may include its own concentration adjustment mechanism, enabling independent control over the amount of each fragrance to be emitted.

In some embodiments, the apparatus may include a blower timing mechanism. This mechanism may be designed to control the operation of each blower in the plurality of blowers. The blower timing mechanism may receive input regarding the desired fragrance combination, the desired intensity of each fragrance, and the desired timing of each fragrance emission. Based on this input, the blower timing mechanism may calculate a fan rotation speed and a fan rotation time for each blower. The fan rotation speed may correspond to the amount of each fragrance to be dispersed from the corresponding cartridge per second, while the fan rotation time may correspond to the duration for which each blower should be on. This feature may allow for precise control over the emission of multiple fragrances, potentially enhancing the overall scent experience in the environment.

In some cases, the blower timing mechanism may employ a machine learning algorithm. This algorithm may be trained using data related to the usage patterns of the apparatus, such as the frequency of each fragrance used, the timing of the scent emissions, and the preferences of the user. Based on this training, the machine learning algorithm may be able to automatically adjust the fan rotation speed and the fan rotation time for each blower, potentially improving the efficiency and accuracy of the fragrance emission.

In some aspects, the machine learning algorithm implemented in the apparatus may be specifically configured to adjust the fan speed and operation time of the blowers based on collected usage data. This adjustment process may enhance the efficiency and effectiveness of scent emission over time.

The usage data collected by the apparatus may include various parameters such as the frequency of use for each fragrance, the duration of each scent emission session, user feedback on scent intensity, and environmental factors such as room size and ambient temperature. In some cases, the apparatus may also collect data on the rate of fragrance depletion from the cartridges.

The machine learning algorithm may analyze this usage data to identify patterns and correlations between different parameters and the perceived effectiveness of scent emission. For example, the algorithm may detect that certain fragrances require higher fan speeds to achieve the desired intensity, while others may be more effectively dispersed at lower speeds.

In some implementations, the algorithm may use techniques such as reinforcement learning to optimize the fan speed and operation time. The algorithm may start with initial settings and then make incremental adjustments based on the feedback received. Positive feedback, such as user satisfaction with scent intensity, may reinforce certain parameter combinations, while negative feedback may prompt the algorithm to explore different settings.

The algorithm may also employ predictive modeling to anticipate the optimal fan speed and operation time for different scenarios. For instance, it may learn that larger rooms typically require longer operation times or higher fan speeds to achieve the desired scent coverage. Similarly, it may adjust these parameters based on the time of day or specific user preferences identified over time.

In some cases, the machine learning algorithm may utilize clustering techniques to group similar usage scenarios and develop optimized settings for each cluster. This approach may allow the apparatus to quickly adapt to different environments or user preferences by applying pre-learned parameter sets.

The algorithm may also incorporate adaptive learning mechanisms to continuously refine its adjustments. As new usage data is collected, the algorithm may update its models and decision-making processes, ensuring that the fan speed and operation time settings remain optimized even as conditions or preferences change over time.

In some implementations, the machine learning algorithm may employ a multi-objective optimization approach, balancing factors such as scent intensity, energy efficiency, and fragrance conservation. For example, it may learn to achieve the desired scent intensity with the lowest possible fan speed and shortest operation time, thereby extending the life of the fragrance cartridges and reducing energy consumption.

The apparatus may also allow users to provide direct feedback on the scent emission experience, which the machine learning algorithm may use to fine-tune its adjustments. This feedback loop may enable the system to adapt to individual preferences more effectively, potentially improving user satisfaction over time.

In some aspects, the machine learning algorithm may develop personalized profiles for different users or environments. These profiles may store optimized fan speed and operation time settings for various fragrances and scenarios, allowing the apparatus to quickly adjust its parameters when switching between different use cases.

The algorithm may also analyze temporal patterns in the usage data to anticipate future needs. For example, it may learn that certain times of day or days of the week typically require stronger scent emission, and proactively adjust the fan speed and operation time accordingly.

In some implementations, the machine learning algorithm may employ ensemble methods, combining multiple models or algorithms to make more robust and accurate adjustments to the fan speed and operation time. This approach may help the system handle a wider range of scenarios and user preferences more effectively.

In other aspects, the apparatus may include different configurations of the scent tubes. For instance, the scent tubes may be arranged in a circular pattern, a linear pattern, or a grid pattern within the housing. The specific arrangement of the scent tubes may depend on various factors, such as the number of fragrances to be emitted, the size and shape of the housing, and the desired aesthetic appearance of the apparatus. Regardless of the specific arrangement, the scent tubes may be designed to securely house the fragrance emitting rods or cartridges and facilitate the controlled emission of scents.

The apparatus for controlled emission of scents may offer several potential advantages. In some cases, the apparatus may provide enhanced control over the intensity, timing, and selection of scents, potentially improving the overall scent experience in various environments. For instance, the apparatus may allow for the precise emission of a single fragrance, the mixing of multiple fragrances, or the scheduling of scent emissions at specific times. This may be particularly beneficial in settings such as retail, hospitality, and residential spaces, where the ambiance can be significantly influenced by the presence of specific scents.

In some aspects, the apparatus may offer improved efficiency in the use of fragrance materials. The apparatus may prevent wastage of fragrance by ensuring that the fragrance is only emitted when the blower is aligned with the scent tube containing the selected fragrance. The apparatus may also prevent leakage of fragrance from the fragrance cartridges when the blower is not in operation, further conserving the fragrance materials. This may result in longer-lasting fragrance cartridges and reduced need for frequent cartridge replacement.

In some cases, the apparatus may provide enhanced convenience and ease of use. The apparatus may include features such as an automated system for inserting fragrance cartridges, a programmable system for autonomous operation, and a remote control mechanism for controlling the apparatus from a distance. These features may reduce the need for manual intervention in the operation of the apparatus, potentially making the apparatus more user-friendly and accessible.

In other aspects, the apparatus may offer potential applications in various fields. For instance, the apparatus may be used in retail stores to create a pleasant shopping environment, in hotels to enhance the guest experience, or in homes to create a relaxing and inviting atmosphere. The apparatus may also be used in therapeutic settings, where specific scents may be used to promote relaxation, reduce stress, or improve mood. The apparatus may also find applications in marketing and branding, where specific scents may be associated with particular brands or products.

In some cases, the apparatus may be designed to be compact and portable, allowing it to be easily moved and positioned in various locations within an environment. The apparatus may also be designed to be aesthetically pleasing, potentially enhancing the decor of the environment in which it is used. The apparatus may be available in various colors, finishes, or designs, allowing it to be customized to match the decor of the environment.

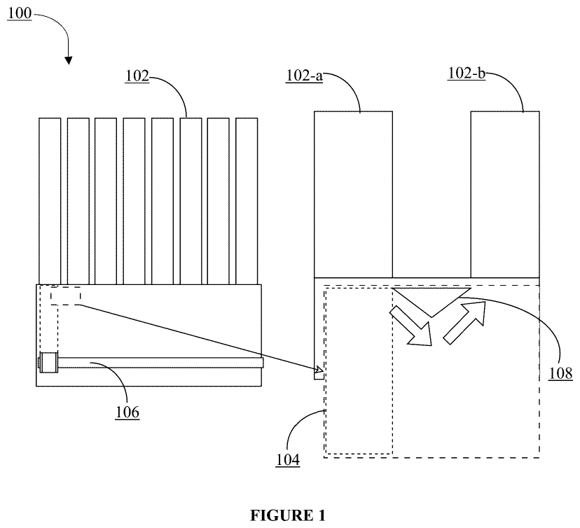

Referring to FIG. 1, the apparatus 100 for controlled emission of scents is depicted. The apparatus 100 may include a set of scent tubes 102, a blower 104, a sliding mechanism 106, and a holding mechanism 108.

In some aspects, the set of scent tubes 102 may be arranged as a series of vertical chambers. Each scent tube, such as scent tube 102-a and scent tube 102-b, may be designed to accommodate fragrance emitting rods or cartridges. The scent tubes 102 may be equidistant from each other and may be carved into the housing of the apparatus 100. In some cases, the scent tubes 102 may have an adjustable radius to accommodate fragrance cartridges of different sizes.

The blower 104 may be positioned adjacent to the set of scent tubes 102. The blower 104 may be designed to push air into one end of a fragrance cartridge, causing the fragrance to be dispersed into the environment through the other end of the cartridge.

The sliding mechanism 106 may be connected to the blower 104, allowing the blower 104 to move horizontally along the set of scent tubes 102. The sliding mechanism 106 may include an adjustment knob that protrudes from the housing of the apparatus 100, enabling manual adjustment of the blower's position. In some cases, the sliding mechanism 106 may include an automatic sliding unit comprising an electronically controlled motor. This motor may receive input from a user interface, such as a set of physical buttons, a graphical user interface, a web interface, or a mobile application, and adjust the position of the blower 104 accordingly.

The holding mechanism 108 may be integrated into the apparatus 100 to ensure precise alignment and secure positioning of the blower 104 during operation. The holding mechanism 108 may include one or more notches or sloping structures that guide the blower 104 to align with a specific scent tube from the set of scent tubes 102. In some embodiments, the holding mechanism 108 may be a magnetic system that pulls the blower 104 towards a position on a predefined track to align the blower 104 with a scent tube from the set of scent tubes 102.

The arrangement of these components within the apparatus 100 may facilitate the selective emission of scents by aligning the blower 104 with a chosen scent tube from the set of scent tubes 102. The sliding mechanism 106 and the holding mechanism 108 may work together to ensure that the blower 104 is correctly positioned for effective scent dispersion.

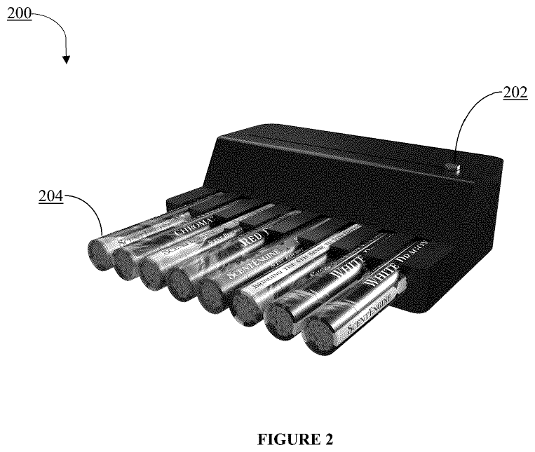

Referring to FIG. 2, a perspective view of the apparatus 200 for controlled emission of scents is depicted. In some aspects, the apparatus 200 may include a housing with a sloped top surface. The housing may serve multiple purposes, such as providing structural support for the internal components of the apparatus 200, protecting these components from external damage, and preventing the loss of fragrance from the fragrance cartridges. The housing may be designed to enclose the entire structure of the apparatus 200, potentially enhancing the durability and longevity of the apparatus 200.

An adjustment knob 202 may be positioned on the top surface of the housing, protruding outward to allow for manual adjustment. The adjustment knob 202 may be connected to the sliding mechanism, enabling the user to manually move the blower along the set of scent tubes. In some cases, the adjustment knob 202 may be designed to provide a comfortable grip for the user, facilitating easy and precise control over the position of the blower.

The front of the apparatus 200 may feature multiple fragrance cartridges 204 arranged in a row. These fragrance cartridges 204 may be partially inserted into the housing, with their ends visible from the front of the apparatus 200. Each fragrance cartridge 204 may contain a different scent, allowing the user to select from a variety of fragrances for emission. In some cases, the fragrance cartridges 204 may be designed to be easily replaceable, enabling the user to change the scents as desired. The arrangement of the fragrance cartridges 204 in a row may facilitate easy access for replacement or maintenance. In some aspects, the fragrance cartridges 204 may be cylindrical in shape, although other shapes may be used in other embodiments. The design of the apparatus 200 may allow for easy access to the fragrance cartridges 204 for replacement or maintenance.

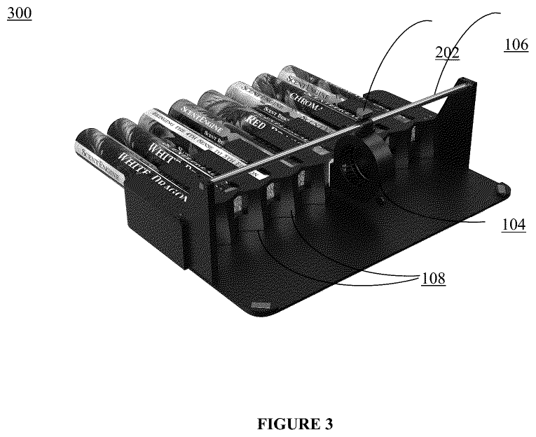

Referring to FIG. 3, a perspective view of the apparatus 300 for controlled emission of scents is depicted. In some aspects, the apparatus 300 may include several components arranged in a compact structure. A blower 104 is positioned at one end of the apparatus 300. Adjacent to the blower 104 is a sliding mechanism 106, which allows for horizontal movement of the blower 104. The sliding mechanism 106 includes an adjustment knob 202 that protrudes from the top of the apparatus 300, enabling manual adjustment of the blower's position.

In some cases, the sliding mechanism 106 may include an automatic sliding unit comprising an electronically controlled motor. This motor may receive input from a user interface, such as a set of physical buttons, a graphical user interface, a web interface, or a mobile application, and adjust the position of the blower 104 accordingly.

A holding mechanism 108 is integrated into the base of the apparatus 300, providing stability and alignment for the blower 104 during operation. The holding mechanism 108 may include one or more notches or sloping structures that guide the blower 104 to align with a specific scent tube from the set of scent tubes 102. In some embodiments, the holding mechanism 108 may be a magnetic system that pulls the blower 104 towards a position on a predefined track to align the blower 104 with a scent tube from the set of scent tubes 102.

The apparatus 300 also includes multiple cylindrical chambers arranged in a row, which are designed to house fragrance cartridges. These components are arranged in a manner that facilitates the controlled emission of scents from the fragrance cartridges through the operation of the blower 104 and its positioning via the sliding mechanism 106.

In some embodiments, the apparatus 300 may also include a fragrance selection mechanism, a locking mechanism for the scent tubes, an automated system for inserting fragrance cartridges, and a leakage prevention mechanism. These additional components may further enhance the functionality and efficiency of the apparatus 300, potentially providing a more sophisticated and accurate method of fragrance dispersion.

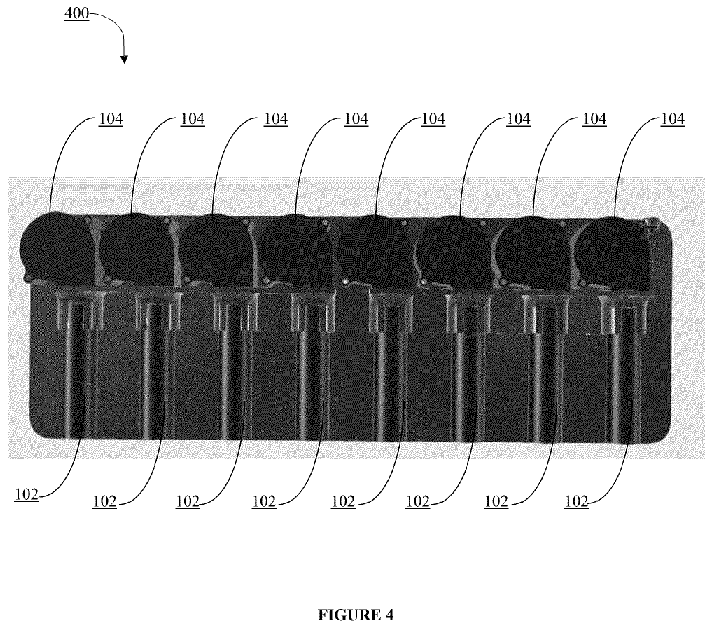

Referring to FIG. 4, an alternate embodiment of the apparatus 400 for controlled emission of scents is depicted. In this embodiment, the apparatus 400 may comprise a plurality of blowers 104, each aligned with an individual scent tube 102. This arrangement may allow for the simultaneous emission of multiple fragrances, potentially enabling the creation of a unique scent blend in the environment.

In some aspects, each blower 104 may be dedicated to a specific scent tube 102, allowing for individual control of scent emission from each tube. This configuration may provide the flexibility to mix one or more fragrances by activating the corresponding blowers 104. The plurality of blowers 104 may be controlled independently, potentially enabling the creation of a wide variety of scent combinations based on user preferences or specific environmental needs.

In some cases, the apparatus 400 may employ a blower timing mechanism to control the operation of each blower 104. This blower timing mechanism may determine when to turn on or off each blower 104 based on user requirements or pre-set schedules. In some embodiments, the blower timing mechanism may employ a machine learning algorithm trained to calculate blower power, on time, and off time based on a required fragrance, required strength of fragrance, and the fragrance cartridges used. This machine learning algorithm may be trained using training data comprising ideal blower power, on time, and off time for a plurality of fragrances, fragrance strengths, and combinations of fragrance cartridges. This may allow for the precise control and customization of scent emission, potentially enhancing the user experience and the overall ambiance of the environment.

In some embodiments, the apparatus 400 may be housed within a rectangular casing, which provides structural support and protection for the internal components. This casing may also prevent the loss of fragrance from the fragrance cartridges, potentially enhancing the efficiency and sustainability of the apparatus 400.

In some aspects, the scent tubes 102 may have an adjustable radius to accommodate fragrance cartridges of different sizes. This adjustability may allow the apparatus 100 to be compatible with a variety of fragrance cartridges, potentially enhancing the versatility and user-friendliness of the apparatus 100. In some cases, the scent tubes 102 may be designed with a locking mechanism to secure a fragrance cartridge in place. This locking mechanism may ensure that the fragrance cartridge is tightly held during operation of the apparatus 100, potentially preventing unwanted movement or dislodgement of the cartridge.

In some embodiments, the apparatus 100 may comprise a leakage prevention mechanism installed in the scent tubes 102. This leakage prevention mechanism may prevent the escape of fragrance from a fragrance cartridge when the blower 104 is not aligned with the fragrance cartridge. This may help to conserve the fragrance material and enhance the efficiency of the apparatus 100. The leakage prevention mechanism may be a rubber membrane, a one-way valve, or a gravity-controlled flap, among other possible designs.

In some cases, the blower 104 may comprise a concentration adjustment mechanism to control the amount of fragrance to be emitted. The concentration adjustment mechanism may be configured to support the blower timing mechanism. This concentration adjustment mechanism may allow for the customization of scent intensity, potentially enhancing the user experience and the overall ambiance of the environment. The concentration adjustment mechanism may receive input regarding the type of fragrance, the concentration of the fragrance, the size of the environment, and the location of the apparatus 100 in the environment. Based on these inputs, the concentration adjustment mechanism may calculate a fan rotation speed for the fan in the blower 104 and a fan rotation time. The fan rotation speed may correspond to the amount of fragrance to be dispersed from the cartridge per second, and the fan rotation time may correspond to the duration for which the blower 104 should be on.

In some embodiments, the apparatus 100 may be programmable for autonomous operation. The apparatus 100 may comprise a processor connected to a memory. The memory may store instructions for various operations of the apparatus 100, such as recording usage data, training a machine learning algorithm based on the usage data, automatically selecting a fragrance and emitting the scent at a particular time of the day based on the training, and receiving feedback from a user to improve the machine learning algorithm. This programmability may allow for the automated and intelligent control of scent emission, potentially enhancing the convenience and efficiency of the apparatus 100.

In some aspects, the apparatus 100 may comprise an automated system for inserting and replacing the fragrance cartridges in the set of scent tubes 102. This automated system may include one or more sensors to measure the quantity of fragrance available in the fragrance cartridge. When the quantity of fragrance falls below a predefined threshold, the automated system may be configured to replace the fragrance cartridge. This replacement process may be facilitated by a set of robotic arms or other mechanical components designed to handle the fragrance cartridges. This automated system may enhance the efficiency of the apparatus 100 and reduce the need for manual intervention in the scent emission process.

In some cases, the blower sliding mechanism 106 may comprise an automatic sliding unit with an electronically controlled motor. This motor may receive input from a user interface, such as a set of physical buttons, a graphical user interface, a web interface, or a mobile application. Based on this input, which may include a fragrance selection or a combination of fragrances, the electronically controlled motor may adjust the position of the blower 104. This automatic sliding unit may enable precise positioning of the blower 104 adjacent to a corresponding scent tube from the set of scent tubes 102, thereby facilitating the controlled emission of a specific scent.

In some embodiments, the blower holding mechanism 108 may be a magnetic system. This magnetic system may include one or more magnets or electromagnets that pull the blower 104 towards a position on a predefined track. This magnetic attraction may help to align the blower 104 with a scent tube from the set of scent tubes 102. The magnetic system may provide a secure and precise positioning of the blower 104, potentially enhancing the accuracy and efficiency of the scent emission process.

In some aspects, the scent tubes 102 may have different shapes, including but not limited to cylindrical, cubical, or rectangular shapes. The shape of the scent tubes 102 may be selected based on various factors, such as the shape of the fragrance cartridges, the available space within the housing 110, or the desired aesthetic design of the apparatus 100. In some cases, the scent tubes 102 may be adjustable to accommodate fragrance cartridges of different shapes and sizes. This adjustability may enhance the versatility of the apparatus 100, allowing it to be compatible with a wide range of fragrance cartridges.

In some aspects, the apparatus 100 for controlled emission of scents may include an integrated webserver control system. This system may embed a webserver directly into the scenting device 100, potentially eliminating the need for separate proprietary control software and hardware tied to a personal computer. Users may access a web application served by the device 100 itself, creating a self-contained, browser-based interface for controlling the apparatus 100.

The integrated webserver control system may enable smartphone interface capabilities. In some cases, the device's 100 embedded webserver may be accessed using any standard smartphone or tablet. This feature may allow for immediate on-site control via a mobile device, potentially reducing complexity and allowing users to interact with the device 100 more conveniently and flexibly, without being tethered to a stationary computer.

In some embodiments, the apparatus 100 may include direct configuration and storage capabilities. The web application may offer functionality for configuring scent intervals and schedules directly within the device's 100 memory. Users may be able to set up daily or hourly schedules, controlling when and how often a scent should be emitted from the scent tubes 102. All scheduling and customization may reside in the device 100, accessible via the mobile interface.

The apparatus 100 may also include features for customizable scent naming and selection. In some cases, the device 100 may allow users to store and recall scents by name. Instead of selecting outputs as generic channels, this approach may support user-assigned scent identifiers or brands directly in the device 100. This feature may simplify selection, reduce guesswork, and ensure rapid adjustment of scent outputs from the scent tubes 102 from a clear, user-friendly interface.

In some aspects, the apparatus 100 may be designed for fully portable, universal access. By leveraging standard web technologies, the system may become platform-agnostic and easily integrate into various environments. It may provide a universal method for users to manage the scenting process-from simple on/off control of the blower 104 to complex timing routines-through a standard web browser, potentially supporting mobility and ease of adoption.

The apparatus 100 may include a processor and memory configured to support these advanced features. In some cases, the processor may be programmed to run the embedded webserver, manage the web application, store user configurations, and control the scent emission based on user inputs received through the web interface. The memory may store the web application files, user configurations, scent names, and scheduling information for controlling the blower 104 and blower sliding mechanism 106.

In some embodiments, the apparatus 100 may include wireless communication capabilities, such as Wi-Fi or Bluetooth, to enable connection with smartphones, tablets, or other devices. This may allow users to access the web application and control the device 100 without the need for physical connections.

The web application interface may be designed to be responsive and adapt to different screen sizes, from smartphones to tablets to desktop computers. In some cases, it may include features such as drag-and-drop scheduling, visual representations of scent intensity and duration, and real-time feedback on the device's 100 status, including the position of the blower 104 and the active scent tube 102.

In some aspects, the apparatus 100 may include security features to protect access to the device and user data. This may include user authentication, encrypted communication between the device 100 and user devices, and the ability to set different levels of access for different users.

The apparatus 100 may also include features for remote monitoring and control. In some cases, authorized users may be able to access the device's 100 web interface from anywhere with an internet connection, allowing for remote adjustments to scent schedules or troubleshooting of the blower 104 or blower sliding mechanism 106.

In some embodiments, the apparatus 100 may include data logging capabilities. It may record usage data, such as which scents were used, when they were emitted, and for how long. This data may be accessible through the web interface, potentially allowing users to analyze patterns and optimize their scent emission strategies using the set of scent tubes 102 and blower 104.

The apparatus 100 may also include integration capabilities with other smart home or building management systems. In some cases, it may be able to communicate with other devices or systems through standard protocols, allowing for coordinated control of scent emission with other environmental factors like lighting or temperature.

In other cases, the apparatus may be designed to be durable and robust, potentially extending its lifespan and reducing the need for frequent maintenance or repair. The apparatus may be made from materials that are resistant to wear and tear, such as metal, plastic, or composite materials. The apparatus may also be designed to withstand various environmental conditions, such as changes in temperature or humidity, further enhancing its durability and longevity.

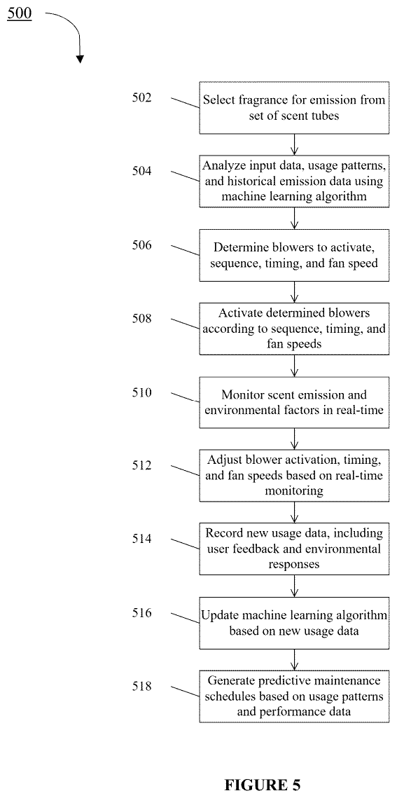

Referring to FIG. 5, a flowchart illustrating a method 500 for controlled emission of scents is depicted. The method 500 may comprise a series of steps that utilize machine learning algorithms to optimize scent emission and enhance user experience.

In some aspects, the method 500 may begin with step 502, where a fragrance is selected for emission from a set of scent tubes. This selection may be made through a user interface or based on pre-programmed settings. Following the fragrance selection, step 504 may involve analyzing input data, usage patterns, and historical emission data using a machine learning algorithm. This analysis may take into account factors such as user preferences, environmental conditions, and previous scent emission performance.

Based on the analysis performed in step 504, step 506 may determine which blowers to activate, along with their sequence, timing, and fan speed. This determination may be made to optimize the scent emission process and create the desired olfactory experience. In step 508, the method may activate the determined blowers according to the specified sequence, timing, and fan speeds.

The method 500 may continue with step 510, which may involve monitoring scent emission and environmental factors in real-time. This monitoring process may utilize sensors integrated into the apparatus to collect data on scent intensity, air quality, and other relevant parameters. Based on this real-time monitoring, step 512 may adjust the blower activation, timing, and fan speeds as needed to maintain optimal scent emission.

In some aspects, the apparatus may employ a real-time feedback loop that integrates with the machine learning algorithm to dynamically adjust blower activation, timing, and fan speeds based on continuous monitoring of scent emission and environmental factors.

The apparatus may include various sensors to collect real-time data on scent concentration, air quality, temperature, humidity, and occupancy levels in the environment. These sensors may provide a continuous stream of data to the machine learning algorithm, allowing for immediate adjustments to the scent emission parameters.

In some implementations, the machine learning algorithm may utilize a sliding window approach to analyze the most recent sensor data. This approach may allow the algorithm to detect short-term fluctuations in environmental conditions and respond promptly. For example, if the sensors detect a sudden increase in room occupancy, the algorithm may increase the fan speed and adjust the blower activation frequency to maintain the desired scent intensity.

The algorithm may employ a multi-layer perceptron neural network to process the real-time sensor data and determine the optimal adjustments to blower parameters. The input layer of this network may receive data from various sensors, while the output layer may provide recommendations for blower activation, timing, and fan speeds. Hidden layers in the network may learn complex relationships between environmental factors and optimal scent emission settings over time.

In some cases, the algorithm may use a combination of rule-based systems and machine learning techniques to make real-time adjustments. For instance, predefined rules may set baseline parameters for different scenarios, while the machine learning component fine-tunes these parameters based on real-time feedback and historical performance data.

The apparatus may implement a proportional-integral-derivative (PID) control system, enhanced by machine learning, to maintain stable scent levels. The machine learning algorithm may continuously adjust the PID controller's parameters based on observed performance, allowing for more precise and adaptive control of the blowers.

In some implementations, the algorithm may use reinforcement learning techniques to optimize blower control in real-time. Each adjustment to blower activation, timing, or fan speed may be treated as an action, with the resulting changes in scent concentration and user feedback serving as rewards or penalties. This approach may allow the system to learn and improve its control strategies over time, adapting to changing conditions and preferences.

The machine learning algorithm may also incorporate predictive elements to anticipate changes in environmental conditions or user preferences. By analyzing patterns in historical data, the algorithm may proactively adjust blower parameters in anticipation of expected changes, such as regular fluctuations in occupancy or temperature throughout the day.

In some aspects, the algorithm may employ ensemble methods, combining the outputs of multiple machine learning models to make more robust decisions about blower adjustments. This approach may help mitigate the impact of any single model's limitations and provide more reliable control across a wide range of scenarios.

The real-time adjustment process may also include a safety check mechanism. Before implementing any changes to blower parameters, the algorithm may evaluate the proposed adjustments against predefined safety and comfort thresholds. This may help prevent excessive scent concentrations or rapid fluctuations that could negatively impact user experience.

In some implementations, the algorithm may use online learning techniques to continuously update its models based on the most recent data. This approach may allow the system to adapt quickly to new scenarios or changes in user preferences without requiring a full retraining of the model.

The apparatus may also include a user feedback mechanism that allows for immediate input on scent intensity or quality. This real-time user feedback may be given high priority in the algorithm's decision-making process, allowing for rapid adjustments to blower parameters to meet user preferences.

In step 514, the method may record new usage data, including user feedback and environmental responses to the scent emission. This data collection may be crucial for improving the system's performance over time. Step 516 may then update the machine learning algorithm based on the newly recorded usage data, potentially enhancing its ability to make accurate predictions and decisions in future scent emission cycles.

In some aspects, the machine learning algorithm implemented in the apparatus may be configured to analyze usage patterns and suggest fragrance combinations and emission schedules based on identified patterns. This functionality may enhance the user experience and optimize scent emission strategies over time.

The algorithm may analyze historical usage data to identify patterns in user preferences, environmental conditions, and scent emission effectiveness. This analysis may include factors such as the frequency of use for different fragrances, popular fragrance combinations, preferred scent intensities at various times of day, and correlations between environmental factors and scent preferences.

In some implementations, the algorithm may employ collaborative filtering techniques to suggest fragrance combinations. By analyzing preferences across multiple users or environments, the system may identify similarities and recommend combinations that have been well-received in similar contexts. For example, if users in similar environments frequently combine lavender and vanilla scents, the algorithm may suggest this combination to new users or in similar settings.

The machine learning algorithm may also utilize time series analysis to identify temporal patterns in scent preferences. This analysis may reveal trends such as preferences for energizing scents in the morning, calming scents in the evening, or seasonal variations in fragrance choices. Based on these identified patterns, the algorithm may suggest emission schedules that align with these temporal preferences.

In some cases, the algorithm may implement a recommendation system that considers both content-based and context-based features. Content-based features may include fragrance notes, intensity levels, and scent categories, while context-based features may encompass environmental factors, time of day, and user activities. By combining these features, the system may generate personalized suggestions for fragrance combinations and emission schedules.

The algorithm may also employ clustering techniques to group similar usage scenarios and develop tailored recommendations for each cluster. For instance, it may identify distinct patterns for weekdays versus weekends, or for different types of environments such as homes, offices, or retail spaces. These clusters may inform more targeted suggestions for fragrance combinations and emission schedules.

In some implementations, the machine learning algorithm may use reinforcement learning to refine its suggestions over time. The system may track user responses to suggested combinations and schedules, treating positive feedback as rewards and negative feedback as penalties. This approach may allow the algorithm to continuously improve its recommendations based on real-world outcomes.

The apparatus may include a user interface that displays suggested fragrance combinations and emission schedules. Users may have the option to accept, modify, or reject these suggestions, providing valuable feedback for further algorithm refinement. In some cases, the interface may also allow users to rate or provide comments on the suggestions, offering more nuanced input for the machine learning model.

In some aspects, the algorithm may incorporate seasonal and event-based factors into its suggestions. For example, it may recommend festive fragrance combinations during holiday seasons or suggest calming scent schedules during typically stressful periods like exam times in educational settings.

The machine learning algorithm may also analyze the effectiveness of past suggestions and adjust its recommendation strategies accordingly. It may track metrics such as user engagement with suggested combinations, adherence to recommended schedules, and subsequent adjustments made by users. This analysis may inform the algorithm's future suggestions, potentially improving their relevance and effectiveness over time.

In some implementations, the algorithm may use natural language processing techniques to analyze user feedback and comments. This analysis may help identify subtle preferences or concerns that users express about different fragrance combinations or emission schedules, allowing for more nuanced and personalized suggestions in the future.

The system may also incorporate a novelty factor in its suggestions, occasionally recommending new or unexpected fragrance combinations to prevent user fatigue and encourage exploration of different scent experiences. The algorithm may carefully balance these novel suggestions with reliable recommendations based on established preferences.

The method 500 may conclude with step 518, where predictive maintenance schedules are generated based on usage patterns and performance data analyzed by the machine learning algorithm. These maintenance schedules may help ensure the longevity and efficient operation of the scent emission apparatus.

In some embodiments, the method 500 may be implemented in a cyclical manner, continuously refining and improving the scent emission process based on accumulated data and user feedback. This iterative approach may allow the system to adapt to changing user preferences, environmental conditions, and other factors that may influence the optimal scent emission strategy.

In some aspects, the apparatus 100 for controlled emission of scents may be implemented in various use cases, enhancing sensory experiences in different environments. Here are example implementations for specific scenarios:

An example scenario may be adding scents to a movie timeline:

In this implementation, the apparatus 100 may be integrated with a movie playback system. The set of scent tubes 102 may contain fragrance cartridges corresponding to various movie-related scents such as blood, gunsmoke, burned rubber, or food smells.

The blower 104 and blower sliding mechanism 106 may be controlled by a processor that receives timing signals from the movie playback system. The processor may interpret these signals and activate the blower 104 to move to the appropriate scent tube 102 at specific points in the movie timeline.

For example, in a horror movie, when a scene involving blood is about to play, the processor may signal the blower sliding mechanism 106 to position the blower 104 adjacent to the scent tube 102 containing the blood scent. The blower 104 may then be activated to disperse the scent into the viewing area.

The blower holding mechanism 108 may ensure that the blower 104 remains correctly aligned with the chosen scent tube 102 during emission, preventing any mixing of scents.

The concentration adjustment mechanism in the blower 104 may control the intensity of the scent based on the scene requirements. For instance, a faint smell of gunsmoke might be emitted for a distant gunfight, while a stronger scent might be used for close-up shooting scenes.

The housing 110 may be designed to blend with the movie theater decor, potentially being integrated into armrests or seat backs for personalized scent delivery.

Another example scenario may be adding scents to a board game:

In this scenario, the apparatus 100 may be connected to a smart board game system. The set of scent tubes 102 may contain fragrances like sulfur, flowers, honey, and tobacco.

The blower sliding mechanism 106 and blower 104 may be controlled by a processor that receives signals from the board game system based on game events or player actions.

For example, when a player's character is about to be attacked by a dragon, the processor may signal the blower sliding mechanism 106 to move the blower 104 to the scent tube 102 containing the sulfur scent. The blower 104 may then emit a brief burst of the scent to enhance the gaming experience.

The blower holding mechanism 108 may ensure quick and accurate alignment with the correct scent tube 102, allowing for rapid transitions between different game events and their corresponding scents.

The leakage prevention mechanism in the scent tubes 102 may be particularly important in this implementation to prevent any unintended mixing of scents that could spoil the gaming experience.

The housing 110 may be designed to resemble a game accessory, potentially themed to match the board game's aesthetic.

Another example scenario may be adding scents to a hospital or recovery process:

In this implementation, the apparatus 100 may be programmed to emit different scents throughout the day to create a more comforting environment for patients.

The set of scent tubes 102 may contain a variety of fragrances such as baked bread, meadow flowers, and cooking aromas. These scents may be carefully selected to avoid any potential allergic reactions or discomfort for patients.

The blower sliding mechanism 106 and blower 104 may be controlled by a processor programmed with a daily schedule. For instance, in the morning, the blower 104 may be positioned next to the scent tube 102 containing the baked bread fragrance.

The concentration adjustment mechanism in the blower 104 may be set to emit a subtle scent, ensuring it's noticeable but not overwhelming for patients with heightened sensitivities.

The blower holding mechanism 108 may secure the blower 104 in place for extended periods, as the scent emissions may occur over longer durations compared to the movie or game scenarios.

The housing 110 may be designed to meet medical facility standards, potentially with antimicrobial coatings and easy-to-clean surfaces.

The apparatus 100 may also include air quality sensors to ensure the scent levels remain within acceptable ranges for a healthcare environment.

In all these implementations, the integrated webserver control system of the apparatus 100 may allow for easy programming and adjustments. Healthcare staff, game masters, or movie theater technicians may use the smartphone interface to modify scent schedules or intensities as needed. The direct configuration and storage capabilities may allow for saving different scent profiles for various movies, games, or patient preferences. The customizable scent naming feature may be particularly useful in these scenarios, allowing for intuitive selection of the right scent for each situation.

A number of implementations have been described. Nevertheless, it will be understood that various modifications may be made without departing from the spirit and scope of the disclosure. Accordingly, other implementations are within the scope of the following claims.

1. An apparatus for controlled emission of scents, comprising:
a housing;
a set of scent tubes within the housing, each scent tube configured to house a fragrance cartridge;
at least one blower configured to blow air towards at least one scent tube of the set of scent tubes; and
a blower timing mechanism configured to control operation of the at least one blower to emit scents, wherein the blower timing mechanism is further configured to determine a fan speed for the at least one blower.

2. The apparatus of claim 1, wherein the at least one blower comprises a plurality of blowers, each blower aligned with a corresponding scent tube of the set of scent tubes.

3. The apparatus of claim 1, further comprising:
a blower sliding mechanism configured to move the at least one blower adjacent to a selected scent tube; and
a blower holding mechanism configured to secure the at least one blower in alignment with the selected scent tube.

4. The apparatus of claim 1, further comprising a fragrance selection mechanism configured to receive user input for selecting a fragrance to be emitted.

5. The apparatus of claim 4, wherein the fragrance selection mechanism comprises at least one of: a set of physical buttons, a graphical user interface, and a microprocessor configured to receive data from at least one of a web interface and a mobile application.

6. The apparatus of claim 1, wherein each scent tube of the set of scent tubes comprises a locking mechanism configured to secure the fragrance cartridge within the scent tube.

7. The apparatus of claim 6, wherein the locking mechanism comprises at least one of: a spring loaded clamp mechanism, a magnetic holder mechanism, a twist and lock mechanism, an adjustable clamp mechanism, a friction fit mechanism, and a bayonet mounting mechanism.

8. The apparatus of claim 1, further comprising a leakage prevention mechanism installed in the set of scent tubes, the leakage prevention mechanism configured to prevent leakage of fragrance from a fragrance cartridge when the corresponding blower is not activated.

9. The apparatus of claim 8, wherein the leakage prevention mechanism comprises at least one of: a rubber membrane, a one-way valve, and a gravity-controlled flap.

10. The apparatus of claim 1, wherein the blower timing mechanism is configured to calculate the fan speed based on at least one of: a type of fragrance, a concentration of the fragrance, a size of an environment, and a location of the apparatus in the environment.

11. The apparatus of claim 1, further comprising a processor connected to a memory, wherein the memory has instructions stored for autonomous operation of the apparatus, including recording usage data and training a machine learning algorithm based on the usage data.

12. The apparatus of claim 11, wherein the machine learning algorithm is configured to adjust the fan speed and operation time for the at least one blower based on the usage data.

13. A method for controlled emission of scents using the apparatus, comprising:
selecting a fragrance for emission from a set of scent tubes housed within a housing;
analyzing input data, usage patterns, and historical emission data using a machine learning algorithm;
determining, based on the analysis, at least one blower to activate, a sequence for activating the determined blowers, a timing for activating the determined blowers, and a fan speed for the determined blowers;
activating the determined blowers according to the determined sequence, timing, and fan speeds to emit the selected fragrance;
monitoring the scent emission and environmental factors in real-time;
adjusting the blower activation, timing, and fan speeds based on the real-time monitoring using the machine learning algorithm;
recording new usage data, including user feedback and environmental responses to the scent emission;
updating the machine learning algorithm based on the new usage data to improve future scent emission decisions; and
generating predictive maintenance schedules for the apparatus based on the usage patterns and performance data analyzed by the machine learning algorithm.

14. The method of claim 13, further comprising:
moving at least one blower adjacent to a selected scent tube using a blower sliding mechanism; and
securing the at least one blower in alignment with the selected scent tube using a blower holding mechanism.

15. The method of claim 13, wherein selecting the fragrance comprises receiving user input through a fragrance selection mechanism, wherein the machine learning algorithm incorporates the user input to personalize future fragrance selections.

16. The method of claim 13, further comprising preventing leakage of fragrance from a fragrance cartridge when the at least one blower is not activated, wherein the machine learning algorithm adjusts leakage prevention based on historical leakage data.

17. The method of claim 13, wherein determining the fan speed is based on a type of fragrance, a size of an environment, a location of the apparatus in the environment, wherein the machine learning algorithm refines these parameters based on performance data collected over time.

18. The method of claim 13, further comprising:
identifying patterns in user preferences and environmental conditions;
suggesting fragrance combinations and emission schedules based on the identified patterns; and
adjusting the scent emission profile in response to detected changes in environmental conditions and user behaviors.

19. A system for controlled emission of scents, comprising:
a housing;
a plurality of scent tubes within the housing, each scent tube configured to house a fragrance cartridge;
at least one blower configured to blow air towards at least one scent tube of the plurality of scent tube; and
a blower timing mechanism configured to control operation of the at least one blower to emit fragrances, wherein the blower timing mechanism is further configured to determine a fan speed for the at least one blower.

20. The system of claim 19, further comprising:
a blower sliding mechanism configured to move the at least one blower adjacent to a selected scent tube; and
a blower holding mechanism configured to secure the at least one blower in alignment with the selected scent tube.
